(19) United States
(12) Patent Application Publication
(10) Pub. No.: US 2026/0166283 A1
(43) Pub. Date: Jun. 18, 2026

(54) ELONGATED MEDICAL APPLIANCE, AND METHOD FOR PRODUCING ELONGATED MEDICAL APPLIANCE

(71) Applicant: ASAHI INTECC CO., LTD., Seto-shi (JP)

(72) Inventors: Soichi FUTAMI, Seto-shi (JP); Yumi TAKATA, Seto-shi (JP); Kazuo SATO, Seto-shi (JP)

(73) Assignee: ASAHI INTECC CO., LTD., Seto-shi (JP)

(21) Appl. No.: 19/414,634

(22) Filed: Dec. 10, 2025

(30) Foreign Application Priority Data
Dec. 12, 2024 (JP) 2024-217534

(51) Int. Cl. A61M 25/09

(52) U.S. Cl. CPC A61M 25/09; A61M 2025/09083; A61M 2025/09108; A61M 2025/09133; A61M 2205/0222

(57) ABSTRACT

An elongated medical appliance includes: an elongated main body part including a first portion and a second portion arranged closer to a proximal end side than the first portion; and a hydrophilic coating layer arranged outside the main body part, and a surface shape of the hydrophilic coating layer located outside the first portion is different from that of the hydrophilic coating layer located outside the second portion. An elongated medical appliance is produced by forming a hydrophilic coating layer outside an elongated main body part including a first portion and a second portion arranged closer to a proximal end side than the first portion such that a surface shape of a portion located outside the first portion is different from that of a portion located outside the second portion. A method for producing an elongated medical appliance, including forming a hydrophilic coating layer outside an elongated main body part.

### TECHNICAL FIELD

The present disclosure relates to an elongated medical appliance and a method for producing an elongated medical appliance.

### BACKGROUND ART

A guide wire is an elongated medical appliance used by being inserted into a living body lumen such as a blood vessel. A guide wire is required to have lubricity in order to improve its operability and to prevent a blood vessel or the like in contact with the guide wire in a living body from being damaged. In order to impart lubricity to a guide wire, conventionally, a surface of the guide wire is coated with a coating material. For example, Patent Document 1 discloses a medical device whose lubricity is adjusted by using different lubricity coatings depending on portions.

### PRIOR ART DOCUMENT

### Patent Document

### Patent Document 1

Japanese Translation of PCT International Application Publication No. JP-T-2009-501056

### SUMMARY

### Technical Problem

An object of the present disclosure is to provide a novel elongated medical appliance whose surface is partially changed in lubricity.

### Solution to Problem

The present disclosure provides, firstly, an elongated medical appliance including: an elongated main body part including a first portion and a second portion arranged closer to a proximal end side than the first portion; and a hydrophilic coating layer arranged outside the main body part, in which a surface shape of the hydrophilic coating layer located outside the first portion is different from a surface shape of the hydrophilic coating layer located outside the second portion.

### BRIEF DESCRIPTION OF DRAWINGS

FIG. 1 is an explanatory view illustrating an overall structure of an elongated medical appliance according to a first embodiment of the present disclosure.

FIG. 2 is an explanatory view illustrating a state of a surface of a first portion of the elongated medical appliance of FIG. 1.

FIG. 3 is an explanatory view illustrating a state of a surface of a second portion of the elongated medical appliance of FIG. 1.

FIG. 4 is an explanatory view illustrating an overall structure of an elongated medical appliance according to a second embodiment of the present disclosure.

### DESCRIPTION OF EMBODIMENTS

Hereinafter, embodiments of the present disclosure will be described with reference to the drawings. The present disclosure is not limited to only the embodiments described below, and the described embodiments are merely examples for describing the technical features of the present disclosure. The shapes and dimensions illustrated in the drawings are merely illustrated to facilitate understanding of the contents of the present disclosure, and do not accurately reflect actual shapes and dimensions.

In the present description, the “distal end side” means a direction along an axial direction of an elongated medical appliance such as a guide wire and a direction in which the elongated medical appliance proceeds toward a target site. The “proximal end side” means a direction along the axial direction of the elongated medical appliance and a direction opposite to the above-described distal end side. The term “distal end” refers to an end portion on the distal end side in any given member or part, and the term “proximal end” refers to an end portion on the proximal end side in any given member or part.

### First Embodiment

FIG. 1 is an explanatory view illustrating an overall structure of a guide wire 100 according to a first embodiment of the present disclosure, and is a longitudinal sectional view taken along an axial direction of the guide wire 100. The guide wire 100 is an example of an elongated medical appliance of the present disclosure, and is used when a combined device such as a catheter or an intracorporeal indwelling device is inserted into a blood vessel, a digestive organ, or the like. The present disclosure is also applicable to an elongated medical appliance other than a guide wire.

The guide wire 100 includes an elongated main body part 10 and a hydrophilic coating layer 6 arranged outside the main body part 10. The main body part 10 includes an elongated core shaft 1 and a coil body 2 arranged outside a distal end portion of the core shaft 1. A distal tip 3 for joining the core shaft 1 and the coil body 2 is provided on the distal end of the guide wire 100, and a fixation portion 4 for fixing the core shaft 1 and the coil body 2 is provided on the proximal end of the coil body 2. A base layer 5 covering the coil body 2 of the main body part 10 is arranged inside the hydrophilic coating layer 6. In the present embodiment, the hydrophilic coating layer 6 and the base layer 5 are formed only on the outer surfaces of the coil body 2 of the main body part 10 and the distal tip 3. The hydrophilic coating layer 6 and the base layer 5 may be formed so as to cover the entire main body part 10 including the fixation portion 4 and the proximal end side of the core shaft 1.

The core shaft 1 is an elongated member serving as a shaft of the guide wire 100. As illustrated in FIG. 1, the core shaft 1 has a small diameter portion 11 on the distal end side and a large diameter portion 13 on the proximal end side, and has a tapered portion 12 between the small diameter portion 11 and the large diameter portion 13, which has an outer diameter gradually decreasing from the proximal end side toward the distal end side. The structure of the core shaft 1 illustrated in FIG. 1 is an example, and the core shaft 1 may have various structures depending on the type, use, and the like of the guide wire 100.

The core shaft 1 can be formed of, for example, materials such as stainless alloys (SUS302, SUS304, SUS316, and the like), superelastic alloys such as an Ni—Ti alloy, a piano wire, a nickel-chromium alloy, a cobalt alloy, and tungsten, but is not limited thereto. The core shaft 1 may be formed of other known materials as long as the core shaft 1 itself can be prevented from being cut and the distal end portion can be rotated.

The small diameter portion 11 has, for example, a flat shape obtained by pressing a columnar material. The large diameter portion 13 has a columnar shape in which the outer diameter is constant from the distal end to the proximal end. The tapered portion 12 has a circular truncated cone shape in which the outer diameter is gradually enlarged from the distal end toward the proximal end so as to connect the small diameter portion 11 to the large diameter portion 13.

The coil body 2 is wound around the core shaft 1 so as to cover the outer periphery of the small diameter portion 11, the tapered portion 12, and a part of the large diameter portion 13 of the core shaft 1. The coil body 2 may be a single coil formed into a hollow cylindrical shape by spirally winding one wire having a circular cross-section, or may be a hollow twisted wire coil formed into a hollow cylindrical shape using a twisted wire that is obtained by twisting a plurality of wires. The coil body 2 may be configured by combining a single coil and a hollow twisted wire coil. Another coil body may be provided inside the coil body 2. The structure of the coil body 2 illustrated in FIG. 1 is an example, and the coil body 2 may have various structures depending on the type, use, and the like of the guide wire 100.

The coil body 2 can be formed of, for example, stainless alloys (SUS302, SUS304, SUS316, and the like), superelastic alloys such as an Ni—Ti alloy, a piano wire, a nickel-chromium alloy, radiotransparent alloys such as a cobalt alloy, and radiopaque alloys such as gold, platinum, tungsten, an alloy containing these elements (for example, a platinum-nickel alloy), but is not limited thereto. The coil body 2 may be formed of known materials other than the above-described materials. In the present embodiment, the coil body 2 is formed as a single member made of the same material, and the outer diameter thereof is configured to be constant from the distal end to the proximal end.

The distal tip 3 for joining the core shaft 1 and the coil body 2 is formed on the distal end of the guide wire 100 (that is, the distal end of the core shaft 1). The distal tip 3 is formed of a metal solder such as a silver solder, a gold solder, zinc, an Sn—Ag alloy, or an Au—Sn alloy, and the distal end of the core shaft 1 and the distal end of the coil body 2 are fixed to each other by the metal solder. The distal tip 3 may be formed of an adhesive such as an epoxy adhesive, and the distal end of the core shaft 1 and the distal end of the coil body 2 may be fixed to each other by the adhesive.

The fixation portion 4 for fixing the core shaft 1 and the coil body 2 is formed on the proximal end of the coil body 2. The fixation portion 4 is formed of a metal solder such as a silver solder, a gold solder, zinc, an Sn—Ag alloy, or an Au—Sn alloy, and the proximal end of the coil body 2 is fixed to the large diameter portion 13 of the core shaft 1 by the metal solder. The fixation portion 4 may be formed of an adhesive such as an epoxy adhesive, and the large diameter portion 13 of the core shaft 1 and the proximal end of the coil body 2 may be fixed to each other by the adhesive.

The base layer 5 is formed of a resin material, and is formed on the outer peripheral surface of the main body part 10 in order to enhance adhesion and integration between the main body part 10 and a hydrophilic coating material forming the hydrophilic coating layer 6 described below. Examples of the resin material for forming the base layer 5 include polyvinyl alcohol (PVA), hydrophilic urethane resins (for example, Hydro Thane (Mitsubishi Chemical Corporation), Hydro MED (Mitsubishi Chemical Corporation), Bionate (DSM), Tecophilic (Lubrizol), and HPU (Dainichiseika Color & Chemicals Mfg. Co., Ltd)), and modified polyolefin resins (for example, polyethylene-acrylic acid (UNITIKA LTD.) and BONDINE (Tokyo Zairyo Co., Ltd.)).

The base layer 5 can be formed by a known coating forming method, for example, by applying the above-described hydrophilic resin material to the outer peripheral surface from the distal tip 3 to the coil body 2. The thickness of the base layer 5 may be, for example, 1 to 1000 μm, but is not limited thereto.

The hydrophilic coating layer 6 is formed of a hydrophilic coating material. The hydrophilic coating layer 6 is usually formed of a material having a higher swellability (hydrophilicity) than the base layer 5. Examples of the hydrophilic coating material include polymers such as hyaluronic acid, polyvinylpyrrolidone and polyethylene glycol, and homopolymers and copolymers obtained by polymerizing monomers such as maleic acid, acrylic acid, methacrylic acid, dimethylacrylamide, carboxybetaine, phosphobetaine, sulfobetaine, methoxyethyl acrylate, hydroxyethyl methacrylate and 2-hydroxypropyl methacrylate.

The hydrophilic coating layer 6 can be formed by a known coating forming method, for example, by applying the above-described hydrophilic coating material to the outer peripheral surface covered with the base layer 5 from the distal tip 3 to the coil body 2. The thickness of the hydrophilic coating layer 6 may be, for example, 0.1 to 20 μm, but is not limited thereto.

In the present embodiment, the main body part 10 includes a first portion 10A and a second portion 10B arranged closer to the proximal end side than the first portion 10A. In the main body part 10, the first portion 10A and the second portion 10B are included in a portion where the coil body 2 is arranged on the outer periphery of the core shaft 1. In the guide wire 100, a surface shape of the hydrophilic coating layer 6 located outside the first portion 10A is different from a surface shape of the hydrophilic coating layer 6 located outside the second portion 10B. In the present embodiment, the hydrophilic coating layer 6 has irregularities on at least a part of the surface, and the surface shape of the hydrophilic coating layer 6 is partially changed by adjusting the degree of the irregularities.

The guide wire 100 is formed such that irregularities on the surface of the hydrophilic coating layer 6 located outside the first portion 10A are larger than irregularities on the surface of the hydrophilic coating layer 6 located outside the second portion 10B. Specifically, the base layer 5 located outside the second portion 10B is formed to be thicker than the base layer 5 located outside the first portion 10A, so that the irregularities on the surface of the hydrophilic coating layer 6 located outside the first portion 10A are made larger than the irregularities on the surface of the hydrophilic coating layer 6 located outside the second portion 10B.

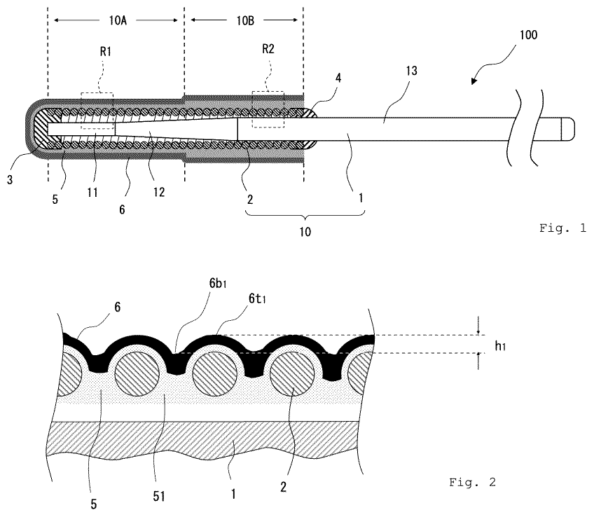

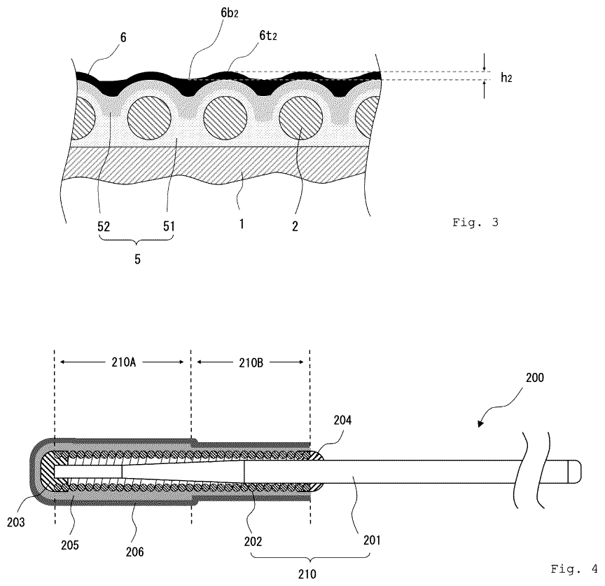

FIG. 2 is an explanatory view illustrating a state of a surface of the first portion 10A of the guide wire 100, and is an enlarged view of the R1 portion illustrated in FIG. 1. FIG. 3 is an explanatory view illustrating a state of a surface of the second portion 10B of the guide wire 100, and is an enlarged view of the R2 portion illustrated in FIG. 1. As illustrated in FIGS. 2 and 3, the hydrophilic coating layer 6 arranged outside the main body part 10 of the guide wire 100 is formed such that the size of the irregularities on the surface of the hydrophilic coating layer 6 located outside the first portion 10A is different from the size of the irregularities on the surface of the hydrophilic coating layer 6 located outside the second portion 10B. The hydrophilic coating layer 6 located outside the first portion 10A of the main body part 10 and the hydrophilic coating layer 6 located outside the second portion 10B of the main body part 10 can be formed to have different surface shapes by changing the size of the irregularities formed on the surface of the hydrophilic coating layer 6, and as a result, the lubricity of the surface can be partially changed.

The size of the irregularities on the surface of the hydrophilic coating layer 6 is a distance (height) from the bottom of a concave portion to the top of a convex portion of the irregularities in a longitudinal section along the axial direction of the guide wire 100. In the first portion 10A (R1 portion) illustrated in FIG. 2, a distance (height) h₁ from a bottom 6b₁ of a concave portion to a top 6t₁ of a convex portion indicates the size of the irregularities in the portion. In the second portion 10B (R2 portion) illustrated in FIG. 3, a distance (height) h₂ from a bottom 6b₂ of a concave portion to a top 6t₂ of a convex portion indicates the size of the irregularities in the portion. The size of the irregularities may be, for example, 1 μm or more, but is not limited thereto. In order to make the hydrophilic coating layer 6 located outside the first portion 10A of the main body part 10 and the hydrophilic coating layer 6 located outside the second portion 10B of the main body part 10 have different surface shapes, the difference between the size h₁ of the irregularities of the first portion 10A (R1 portion) and the size h₂ of the irregularities of the second portion 10B (R2 portion) may be, for example, 100 nm or more, or 200 nm or more, but is not limited thereto.

In the present embodiment, the thickness of the base layer 5 located outside the first portion 10A is made different from the thickness of the base layer 5 located outside the second portion 10B, so that making the size of the irregularities on the surface of the hydrophilic coating layer 6 located outside the first portion 10A different from the size of the irregularities on the surface of the hydrophilic coating layer 6 located outside the second portion 10B is achieved. By forming the base layer 5 covering the coil body 2 such that the portion located outside the first portion 10A and the portion located outside the second portion 10B have different thicknesses, the size of the irregularities formed on the surface of the hydrophilic coating layer 6 formed on the base layer 5 can be changed.

When the base layer 5 is formed on the outer peripheral surface of the coil body 2, the base layer 5 is formed in a shape following the shape of the coil body 2. That is, the base layer 5 has irregularities corresponding to the outer shape of the coil body 2. When the base layer 5 is made thick, the irregularities exposed on the surface of the base layer 5, which are caused by the shape of the coil body 2, become small, and when the base layer 5 is made thin, the irregularities exposed on the surface of the base layer 5, which are caused by the shape of the coil body 2, become large. When the irregularities on the surface of the base layer 5 become small, the irregularities on the surface of the hydrophilic coating layer 6 formed on the base layer 5 also become small. As the surface of the hydrophilic coating layer 6 becomes flatter and smoother, the resistance becomes smaller and the lubricity becomes higher. When the irregularities on the surface of the base layer 5 become large, the irregularities on the surface of the hydrophilic coating layer 6 formed on the base layer 5 also become large. As the surface of the hydrophilic coating layer 6 becomes rougher, the resistance becomes larger and the lubricity becomes lower.

In the guide wire 100 of the present embodiment, the base layer 5 located outside the first portion 10A has a single-layer structure, and the base layer 5 located outside the second portion 10B has a double-layer structure, so that the thickness of the base layer 5 is made partially different. As illustrated in FIG. 2, the base layer 5 located outside the first portion 10A has a single-layer structure of only a first base layer 51, and the hydrophilic coating layer 6 is formed on the first base layer 51. As illustrated in FIG. 3, the base layer 5 located outside the second portion 10B has a double-layer structure of the first base layer 51 and a second base layer 52, the second base layer 52 is formed on the first base layer 51, and the hydrophilic coating layer 6 is formed on the second base layer 52. Due to the difference in the thickness of the base layer 5, the size h₁ of the irregularities of the first portion 10A (R1 portion) illustrated in FIG. 2 is larger than the size h₂ of the irregularities of the second portion 10B (R2 portion).

The thickness of the first base layer 51 may be, for example, 0.5 to 500 μm, but is not limited thereto. The thickness of the second base layer 52 may be, for example, 0.5 to 500 μm, but is not limited thereto. The first base layer 51 and the second base layer 52 may be formed of the same hydrophilic resin material, or may be formed of different hydrophilic resin materials.

In such a guide wire 100, the lubricity of the hydrophilic coating layer 6 arranged outside the second portion 10B located on the proximal end side of the main body part 10 can be made more excellent than the lubricity of the hydrophilic coating layer 6 arranged outside the first portion 10A located on the distal end side of the main body part 10. Since the guide wire 100 achieved in this way has characteristics in that the distal end side does not slide too much and the proximal end side has sufficient lubricity, the guide wire 100 is suitable for, for example, a guide wire for PTCA (percutaneous transluminal coronary angioplasty) treatment in which the proximal end portion of the coil body 2 is required to have lubricity for safety while the distal end portion is required to reduce lubricity so as to suppress perforation of a blood vessel and transmit the feeling of manipulation to the professional's hand.

According to the guide wire 100 as described above, even when the hydrophilic coating layer 6 is formed of one material, the lubricity of the surface can be partially changed by a simple method in which the hydrophilic coating layer 6 located outside the first portion 10A of the main body part 10 and the hydrophilic coating layer 6 located outside the second portion 10B of the main body part 10 are formed to have different surface shapes.

The guide wire 100 of the present embodiment can be produced by, for example, the following method.
(1) An elongated main body part 10 including a core shaft 1 and a coil body 2 arranged outside a distal end portion of the core shaft 1 is prepared. The main body part 10 includes a first portion 10A and a second portion 10B arranged closer to a proximal end side than the first portion 10A in a portion where the coil body 2 is arranged.
(2) A first base layer 51 is formed so as to cover the coil body 2 of the main body part 10. The first base layer 51 is formed on the outer peripheral surface of both the first portion 10A and the second portion 10B of the coil body 2 of the main body part 10.
(3) After forming the first base layer 51, a second base layer 52 is formed only on a portion located outside the second portion 10B of the coil body 2 of the main body part 10. As a result, a base layer 5 having a single-layer structure is formed outside the first portion 10A of the coil body 2 of the main body part 10, and a base layer 5 having a double-layer structure is formed outside the second portion 10B of the coil body 2 of the main body part 10.
(4) After the base layer 5 is formed so as to cover the coil body 2 of the main body part 10, a hydrophilic coating layer 6 is formed so as to cover the base layer 5. Since the thickness of the base layer 5 located outside the first portion 10A is different from the thickness of the base layer 5 located outside the second portion 10B, the hydrophilic coating layer 6 is formed such that the surface shape of a portion located outside the first portion 10A is different from the surface shape of a portion located outside the second portion 10B. Specifically, the hydrophilic coating layer 6 is formed such that the size of the irregularities on the surface of the hydrophilic coating layer 6 located outside the first portion 10A is different from the size of the irregularities on the surface of the hydrophilic coating layer 6 located outside the second portion 10B.

### Second Embodiment

FIG. 4 is an explanatory view illustrating an overall structure of a guide wire 200 according to a second embodiment of the present disclosure. The guide wire 200 includes an elongated main body part 210 and a hydrophilic coating layer 206 arranged outside the main body part 210. The main body part 210 includes an elongated core shaft 201 and a coil body 202 arranged outside a distal end portion of the core shaft 201. A distal tip 203 for joining the core shaft 201 and the coil body 202 is provided on the distal end of the guide wire 200, and a fixation portion 204 for fixing the core shaft 201 and the coil body 202 is provided on the proximal end of the coil body 202. A base layer 205 covering the coil body 202 of the main body part 210 is arranged inside the hydrophilic coating layer 206.

Since the structures of the main body part 210 including the core shaft 201 and the coil body 202, the distal tip 203, and the fixation portion 204 are the same as those of the respective constituent elements (the main body part 10 including the core shaft 1 and the coil body 2, the distal tip 3, and the fixation portion 4) of the guide wire 100 of the first embodiment, detailed description thereof will be omitted. Since the base layer 205 and the hydrophilic coating layer 206 are formed in the same manner as the base layer 5 and the hydrophilic coating layer 6 of the guide wire 100 of the first embodiment, detailed description thereof will be omitted.

Unlike the guide wire 100 of the first embodiment, the guide wire 200 is formed such that irregularities on the surface of the hydrophilic coating layer 206 located outside a first portion 210A are smaller than irregularities on the surface of the hydrophilic coating layer 206 located outside a second portion 210B. Specifically, the base layer 205 located outside the first portion 210A is formed to be thicker than the base layer 205 located outside the second portion 210B, so that the irregularities on the surface of the hydrophilic coating layer 206 located outside the first portion 210A are made smaller than the irregularities on the surface of the hydrophilic coating layer 206 located outside the second portion 210B. In the first embodiment, the base layer 5 located outside the first portion 10A has a single-layer structure of only the first base layer 51, and the base layer 5 located outside the second portion 10B has a double-layer structure of the first base layer 51 and the second base layer 52. However, in the guide wire 200 of the second embodiment, the base layer 205 located outside the first portion 210A has a double-layer structure, and the base layer 205 located outside the second portion 210B has a single-layer structure, so that the thickness of the base layer 205 is made partially different. The specific configuration of the base layer 205 is the same as the configuration of the base layer 5 described in the first embodiment except that the first portion 10A and the second portion 10B are replaced with each other, and thus the detailed description thereof will be omitted.

In such a guide wire 200, the lubricity of the hydrophilic coating layer 206 arranged outside the first portion 210A located on the distal end side of the main body part 210 can be made more excellent than the lubricity of the hydrophilic coating layer 206 arranged outside the second portion 210B located on the proximal end side of the main body part 210. Since the guide wire 200 achieved in this way has characteristics in that the distal end side slides well and the proximal end side does not slide too much, the guide wire 200 is suitable for, for example, a guide wire required to pass through a stent strut.

The present disclosure provides, firstly, an elongated medical appliance including: an elongated main body part including a first portion and a second portion arranged closer to a proximal end side than the first portion; and a hydrophilic coating layer arranged outside the main body part, in which a surface shape of the hydrophilic coating layer located outside the first portion is different from a surface shape of the hydrophilic coating layer located outside the second portion (Disclosure 1).

According to the disclosure (Disclosure 1), even when the hydrophilic coating layer is formed of one material, the elongated medical appliance whose surface is partially changed in lubricity can be easily produced only by forming the hydrophilic coating layer located outside the first portion of the main body part and the hydrophilic coating layer located outside the second portion of the main body part to have different surface shapes.

In the above-described disclosure (Disclosure 1), the hydrophilic coating layer has irregularities on at least a part of a surface, and a size of the irregularities on the surface of the hydrophilic coating layer located outside the first portion may be different from a size of the irregularities on the surface of the hydrophilic coating layer located outside the second portion (Disclosure 2).

According to the disclosure (Disclosure 2), the hydrophilic coating layer located outside the first portion of the main body part and the hydrophilic coating layer located outside the second portion of the main body part can be formed to have different surface shapes by changing the size of the irregularities formed on the surface of the hydrophilic coating layer, and as a result, the lubricity of the surface can be partially changed.

In the above-described disclosure (Disclosure 2), the main body part includes a core shaft and a coil body arranged outside a distal end portion of the core shaft, and the first portion and the second portion may be included in a portion where the coil body is arranged (Disclosure 3). In the above-described disclosure (Disclosure 3), a base layer covering at least the coil body of the main body part is arranged inside the hydrophilic coating layer, and a thickness of the base layer located outside the first portion may be different from a thickness of the base layer located outside the second portion (Disclosure 4).

According to the disclosures (Disclosures 3 and 4), by forming the base layer covering the coil body such that the portion located outside the first portion and the portion located outside the second portion have different thicknesses, the size of the irregularities formed on the surface of the hydrophilic coating layer formed on the base layer can be changed.

In the above-described disclosures (Disclosures 2 to 4), the irregularities on the surface of the hydrophilic coating layer located outside the first portion may be formed to be larger than the irregularities on the surface of the hydrophilic coating layer located outside the second portion (Disclosure 5).

According to the disclosure (Disclosure 5), the elongated medical appliance, in which the lubricity of the hydrophilic coating layer arranged outside the second portion located on the proximal end side of the main body part is more excellent than the lubricity of the hydrophilic coating layer arranged outside the first portion located on the distal end side of the main body part, can be obtained. Therefore, the elongated medical appliance having characteristics in that the distal end side does not slide too much and the proximal end side has sufficient lubricity can be achieved.

In the above-described disclosures (Disclosures 2 to 4), the irregularities on the surface of the hydrophilic coating layer located outside the first portion may be formed to be smaller than the irregularities on the surface of the hydrophilic coating layer located outside the second portion (Disclosure 6).

According to the disclosure (Disclosure 6), the elongated medical appliance, in which the lubricity of the hydrophilic coating layer arranged outside the first portion located on the distal end side of the main body part is more excellent than the lubricity of the hydrophilic coating layer arranged outside the second portion located on the proximal end side of the main body part, can be obtained. Therefore, the elongated medical appliance having characteristics in that the distal end side slides well and the proximal end side does not slide too much can be achieved.

The present disclosure provides, secondly, a method for producing an elongated medical appliance, including: forming a hydrophilic coating layer outside an elongated main body part including a first portion and a second portion arranged closer to a proximal end side than the first portion such that a surface shape of a portion located outside the first portion is different from a surface shape of a portion located outside the second portion (Disclosure 7).

According to the disclosure (Disclosure 7), even when the hydrophilic coating layer is formed of one material, the elongated medical appliance whose surface is partially changed in lubricity can be produced by a simple method by forming the hydrophilic coating layer located outside the first portion of the main body part and the hydrophilic coating layer located outside the second portion of the main body part to have different surface shapes.

In the above-described disclosure (Disclosure 7), the hydrophilic coating layer is formed to have irregularities on at least a part of a surface, and the hydrophilic coating layer may be formed such that a size of the irregularities on the surface of the hydrophilic coating layer located outside the first portion is different from a size of the irregularities on the surface of the hydrophilic coating layer located outside the second portion (Disclosure 8).

According to the disclosure (Disclosure 8), the hydrophilic coating layer located outside the first portion of the main body part and the hydrophilic coating layer located outside the second portion of the main body part can be formed to have different surface shapes by changing the size of the irregularities formed on the surface of the hydrophilic coating layer, and as a result, the lubricity of the surface can be partially changed.

In the above-described disclosure (Disclosure 8), the main body part includes a core shaft and a coil body arranged outside a distal end portion of the core shaft, the first portion and the second portion may be included in a portion where the coil body is arranged, and, before the hydrophilic coating layer is formed, a base layer may be formed so as to cover the coil body of the main body part (Disclosure 9).

According to the disclosure (Disclosure 9), by forming the base layer covering the coil body such that the portion located outside the first portion and the portion located outside the second portion have different thicknesses, the size of the irregularities formed on the surface of the hydrophilic coating layer formed on the base layer can be changed.

In the above-described disclosure (Disclosure 9), the base layer includes a first base layer and a second base layer, the first base layer is formed in both the first portion and the second portion, and the second base layer is formed outside the first base layer in one of the first portion and the second portion (Disclosure 10).

According to the disclosure (Disclosure 10), since the thickness of the base layer covering the coil body can be partially changed by forming the base layer to have a double-layer structure partially, the size of the irregularities formed on the surface of the hydrophilic coating layer formed on the base layer can be changed by a simple method.

The elongated medical appliance according to the present disclosure has been described above with reference to the drawings. The present disclosure is not limited to the above-described embodiments, and various modifications can be made. For example, the lubricity may be changed at three or more positions on the surface of the hydrophilic coating layer depending on the use of the elongated medical appliance.

What is claimed is:

1. An elongated medical appliance comprising:
an elongated main body part including a first portion and a second portion arranged closer to a proximal end side than the first portion; and
a hydrophilic coating layer arranged outside the main body part, wherein
a surface shape of the hydrophilic coating layer located outside the first portion is different from a surface shape of the hydrophilic coating layer located outside the second portion.

2. The elongated medical appliance according to claim 1, wherein
the hydrophilic coating layer has irregularities on at least a part of a surface, and
a size of the irregularities on the surface of the hydrophilic coating layer located outside the first portion is different from a size of the irregularities on the surface of the hydrophilic coating layer located outside the second portion.

3. The elongated medical appliance according to claim 2, wherein
the main body part includes a core shaft and a coil body arranged outside a distal end portion of the core shaft, and
the first portion and the second portion are included in a portion where the coil body is arranged.

4. The elongated medical appliance according to claim 3, wherein
a base layer covering at least the coil body of the main body part is arranged inside the hydrophilic coating layer, and
a thickness of the base layer located outside the first portion is different from a thickness of the base layer located outside the second portion.

5. The elongated medical appliance according to claim 2, wherein
the irregularities on the surface of the hydrophilic coating layer located outside the first portion are formed to be larger than the irregularities on the surface of the hydrophilic coating layer located outside the second portion.

6. The elongated medical appliance according to claim 2, wherein
the irregularities on the surface of the hydrophilic coating layer located outside the first portion are formed to be smaller than the irregularities on the surface of the hydrophilic coating layer located outside the second portion.

7. A method for producing an elongated medical appliance, comprising:
forming a hydrophilic coating layer outside an elongated main body part including a first portion and a second portion arranged closer to a proximal end side than the first portion such that a surface shape of a portion located outside the first portion is different from a surface shape of a portion located outside the second portion.

8. The method for producing an elongated medical appliance according to claim 7, wherein
the hydrophilic coating layer is formed to have irregularities on at least a part of a surface, and
the hydrophilic coating layer is formed such that a size of the irregularities on the surface of the hydrophilic coating layer located outside the first portion is different from a size of the irregularities on the surface of the hydrophilic coating layer located outside the second portion.

9. The method for producing an elongated medical appliance according to claim 8, wherein
the main body part includes a core shaft and a coil body arranged outside a distal end portion of the core shaft,
the first portion and the second portion are included in a portion where the coil body is arranged, and,
before the hydrophilic coating layer is formed, a base layer is formed so as to cover the coil body of the main body part.

10. The method for producing an elongated medical appliance according to claim 9, wherein
the base layer includes a first base layer and a second base layer,
the first base layer is formed in both the first portion and the second portion, and
the second base layer is formed outside the first base layer in one of the first portion and the second portion.
